(19) United States
(12) Patent Application Publication
(10) Pub. No.: US 2021/0292058 A1
(43) Pub. Date: Sep. 23, 2021

(54) SPINNING LID WITH CENTER BOWL CONTAINMENT FOR STORAGE

(71) Applicant: Bryan Smith, Midland, MI (US)

(72) Inventor: Bryan Smith, Midland, MI (US)

(21) Appl. No.: 17/249,804

(22) Filed: Mar. 15, 2021

Related U.S. Application Data
(60) Provisional application No. 62/991,212, filed on Mar. 18, 2020.

(51) Int. Cl. B65D 51/18; A45C 11/00; A45C 13/02

(52) U.S. Cl. CPC B65D 51/18; A45C 11/00; A45C 13/02; A61F 17/00; B65D 2251/0018; B65D 2251/0081; A45C 2011/007

(57) ABSTRACT

A storage container includes a base having a plurality of sections and defining a center chamber and a spinning lid disposed on the base. The spinning lid defines an aperture. An intermediate lid is disposed between the base and the spinning lid. The intermediate lid defines an opening. The center chamber, aperture, and opening are coaxially aligned to define a receptacle.

### CROSS-REFERENCE TO RELATED APPLICATIONS

This patent application is a non-provisional filing of and claims priority to, and all advantages of, U.S. Provisional Patent Application No. 62/991,212 filed on Mar. 18, 2020 and titled “SPINNING LID WITH CENTER BOWL CONTAINMENT FOR STORAGE,” the contents of which are incorporated herein by reference in their entirety.

### BACKGROUND

Storage containers make it easy to organize and carry small items. For example, first aid kits make bandages, antibiotics, tape, medication, and other items easily accessible during an emergency. First aid kits come in various sizes. Some first aid kits may hold a few bandages. Others may hold larger items that are useful to treat a more serious injury. First aid kits can be found in homes, offices, workplaces, schools, vehicles, gym bags, backpacks, and purses. The contents of the first aid kit can be replenished as needed.

### BRIEF DESCRIPTION OF THE DRAWINGS

FIG. 1 illustrates an example storage container.

FIG. 2 is a perspective view of an example base used with the storage container of FIG. 1.

FIG. 3A is a top perspective view of an example intermediate lid used with the storage container of FIG. 1.

FIG. 3B is a bottom perspective view of the example intermediate lid of FIG. 3A.

FIG. 4A is a top perspective view of an example spinning lid used with the storage container of FIG. 1.

FIG. 4B is a bottom view of the spinning lid of FIG. 4A.

FIG. 5 is a perspective view of an example trash cover used with the storage container of FIG. 1.

FIG. 6 is an exploded view of the storage container of FIG. 1.

FIG. 7 is a cross-sectional view of the storage container of FIG. 1 taken across the line A-A.

### DETAILED DESCRIPTION

Storage containers seek to provide users access to stored items in a convenient manner. Storage containers fail to meet the user's needs if they are cumbersome, confusing, disorganized, slow for the user to operate, or hard for the user to operate. For example, first aid kits are often square or rectangular containers with one lid, lids within lids, or zippers. The same is often true for other types of storage containers and organizers. The awkwardness of first aid kits makes it difficult for users to quickly find the first aid item needed. Furthermore, existing containers typically do not have a designated area for each particular item. Items, therefore, can get mixed together upon the initial opening of the storage container. Moreover, as is the case with other storage containers, items stored in existing first aid kits can fall out or be hard to locate when the user is looking for a particular item, such as ointment, at a particular moment of need. The urgency of needing items from a first aid kit can further frustrate the user of a disorganized first aid kit. That problem is compounded by allowing items to mix within the storage container, especially if the storage container is to be transported. For example, wrappers and other refuse can accumulate which further inhibits the user from quickly finding what he or she needs.

The disclosed concept provides a systematic approach to organizing items into a storage container that is intuitive to the user and provides an independent center bowl or cup feature built into the spinning lid that is unique to a storage container and can be used as appropriately by the user separate from the base storage. Applicable use of this storage container technology can be any of the following but is not limited to this list: First aid storage, pill organizer, lunch or snack organizer, etc.

In one possible implementation, the storage container can provide step-by-step instructions and the supplies for each step in the order in which the supplies are used. In the context of a first aid device, the storage container can help a user quickly administer first aid because it can make the steps and supplies for, e.g., cleaning and treating a minor wound readily available.

Also, the storage container contains a bowl in the center of the lid which is always present and independent of the base storage. In the case of a first aid kit, the bowl can act as a refuse section that allows the user to easily discard wrappers and other debris involved with first aid treatment so that the material does not linger, distract, or otherwise slow down the user during or after the process of administering first aid. The device is easily identifiable due to its generally circular shape and can be stored in readily accessible areas such as but not limited to a purse, glove box, gym bag, drawer, cabinet, etc.

The disclosed storage container contains the items to store and organize different material items and make them available to a user in a systematic fashion to hold items such as but not limited to first aid, food, nails, screws, etc. The storage container includes the following components: a base and a rotating spinning lid. These components are connected so that the spinning lid attaches to the base and the spinning lid sits overtop of the base. The rotating spinning lid covers the base top and could cover at least a portion of the base wall. The spinning lid may spin freely around the base while the base is stationary, or vice versa. Furthermore, the spinning lid may have a cup- or bowl-shaped indent, or an opening for one, in the center acting as an independent containment area. The spinning lid can conceal the majority of the base, so it strategically exposes only a portion of the base at a time. In doing so, a user may be able to access only a portion of the items in the base at a time.

The spinning lid may define an opening, such as but not limited to a cutout or hole in its surface, which will expose or reveal only a portion of the base as the spinning lid rotates about the base. The opening exposes a particular section of the bases and therefore allows a user to access that section of the base and the items contained within it. Furthermore, the spinning lid defines a center bowl that acts as a separate containment area which is independent of the base and available to receive refuse regardless of the position of the spinning lid. In some possible approaches, the center bowl within the spinning lid is open at the bottom so refuse falls through the center bowl and into a center of the base. The center of the base in this example is not affected by the spinning lid's placement.

The base can store any items such as, but not limited to, first aid items, food, beads, etc., and the rotating spinning lid allows a user to systematically access the items within the base depending on the rotated position of the spinning lid. Furthermore, the center of the base includes a receptacle defining a cavity for receiving the center bowl of the spinning lid. The receptacle may have any configuration such as a generally cylindrical configuration generally matching that of the center bowl of the spinning lid. When the center bowl of the spinning lid is inserted into the receptacle, the spinning lid may spin relative to the base. Moreover, other points of connection between the base and spinning lid may be used to fix the spinning lid vertically or horizontally relative to the base without inhibiting the rotation of the spinning lid relative to the base. For instance, buttons, snaps, stems, clips, or the like may be used to connect the base and spinning lid without inhibiting rotation of the two components. Furthermore, the type of connection can be selected to allow the base and spinning lid to be permanently or removably attached.

The base includes a hollow cylindrical container piece with a center connection component that connects to the spinning lid. The center connection component may be a hollow cylinder that receives and connects to the spinning lid. Furthermore, the base may have a bottom, sides and a top which allows for the interior to contain one or more chambers which may contain items selected by the end user based on the user's intended use of the storage container. Examples of such items may include bandages, gauze, or antibiotic ointment when the storage container is used as a first aid kit.

The chambers may be accessed via openings in the base, located at, but not limited to, the side of the base or the top of the base and may or may not contain features such as but not limited to doors, holes, latches or other features to similarly expose or open and/or close the chambers. Also, the storage container may have one or more walls within the chamber of the base in order to separate the material items within the base. The base may also contain an exposed tacky material or tacky grip around the bottom area and the lower edge exterior walls. The tacky material may be made of but, is not limited to, rubber, silicone, or another tacky material. Furthermore, the tacky grip may extend past the sides of the base and slightly up the exterior wall of the base and covering all or some of the base bottom. Exposing the tacky material to the base wall just below the spinning lid may allow a user to grip the device at the tacky location to keep the base stationary while spinning the spinning lid. Similarly, the tacky material on the base may provide enough grip so that the base remains stationary during rotation of the spinning lid while it's on a surface such as a table or counter. Furthermore, the chambers may or may not have a secondary access point for refilling of items when depleted. The access point for this secondary access to the items can be but is not limited to the bottom, top, or sides. The access points for the items may be a removable or permanent base lid separate from the spinning lid. The base lid may be attached to the base either from the top or bottom and conceal the items inside thus completing the base's storage functionality. In some possible approaches, the base may be made out of a rigid material such as, but not limited to, plastic, resin, metal, wood or other materials.

The spinning lid may include a circular disc with a center bowl or cavity. In some possible implementations, the spinning lid includes sides. The spinning lid may connect to the top of the base at the center of the spinning lid at its bowl feature with a connection component such as, but not limited to, a snap, pin or other fastener. The spinning lid may have a generally flat circular shape with or without sides and containing a center bowl or cavity. The center bowl of the spinning lid may serve the purpose of additional storage outside the base container and it is not dependent on the position of the spinning lid. Also, the center bowl may be the primary connection point with the base, although additional or alternative connections may be possible around the disc part of the lid or the sides. Furthermore, the cavity in the center of the spinning lid may be open or have a lid feature. In some instances, the spinning lid may cover any portion of the base top and sides, such as but not limited so that the sides of the spinning lid extend down just before it meets the tacky material found on the outer bottom edge of the base's side. A tacky grip, or other similar feature such as but not limited to bumps or slats, on the spinning lid may allow a user to grip and control the spin of the lid. These tacky grips can be located on the top or sides of the spinning lid. Nodules may resist or stop the rotation of the spinning lid at certain points around the base without completely inhibiting rotation of the spinning lid relative to the base. The nodules may allow the spinning lid to come to a comfortable stop at certain points to make the items stored in the base easily accessible. Furthermore, the spinning lid may be fixed or separable relative to the base. If separably, the spinning lid may be manually reattached to the base by the user. The spinning lid may be made out of a rigid material such as, but not limited to, plastic, resin, metal, wood or other materials.

The storage container provides an intuitive and user-friendly systematic approach to organize while allowing the user to extract stored items. The storage container provides a rotating lid which aides in the use of and contains an independent storage component, separate from the storage items found in the base of the device allowing for additional storage dynamic.

The storage container includes a (1) spinning or rotating lid with an inset bowl, cup or otherwise described as but not limited to a cavity in which the said spinning lid acts as a lid and covers a base container; (2) the spinning lid has the presence of containment in of itself due to its center cup feature, outside of the base containment system; (3) the spinning lid and connection there into a base container allows for this invented device to systematically and intuitively allow a user to extract the items of the device in a methodical fashion; (4) the built in cup within the spinning lid adds additional storage apart from the base containment that is independent and always available to the user no matter the spun position of the lid.

The elements shown may take many different forms and include multiple and/or alternate components and facilities. The example components illustrated are not intended to be limiting. Indeed, additional or alternative components and/or implementations may be used. Further, the elements shown are not necessarily drawn to scale unless explicitly stated as such.

The storage container includes a spinning lid with an inset center bowl container in-which the spinning lid acts as a lid and covers a base container. The following is an example of the storage container to store items and provide access to the items in a systematic and user-friendly way. This specific design, use of material, manufacturing technique, etc. does not encompass all possibilities to utilize a spinning lid with center cup feature which attaches to a base container.

When used as a medical first aid container, the storage container may include a hollow circular base container including a floor, outer walls, a center hollow cylinder with the same floor as the main base and chamber dividing walls between the exterior walls of the center cylinder and the inside of the outer walls. The first aid kit may have a permanent base lid with a center hole; the lid may be permanently attached to the top of the base once the items are inserted during assembly but before the spinning lid is attached. Also, there may be a spinning lid that includes a flat circular top with a bowl feature at the center. The spinning lid may extend to the edge of the base container and then drape to cover almost the entire side wall of the base. This spinning lid may connect at the center to the base permanently, thus when attached the majority of the spinning lid may be slightly above the base container in all areas except the center connection location and other locations designed to inhibit spinning

The hollow circular base may be formed of a rigid material such as plastic and may be approximately 4.5 inches in diameter. The thickness of the plastic may be relatively uniform across all parts. For purposes of illustration, the thickness may be about one-sixteenth of an inch. The height of the exterior walls may be approximately one inch tall. The inner hollow cylinder may also be the same thickness and height as the exterior walls and may be approximately 1.5 inches in diameter. The center cylinder walls in this particular approach may have a slight taper or reduction in circumference from top to bottom of approximately one-fourth inch from top to bottom in circumference size. At the lower portion of the cylinder where it meets the base floor, the interior of the cylinder walls may be undercut, leaving an inward sloping side and leaving an overhanging edge. This overhanging edge may be how the spinning lid component may attach to the base and still be allowed to spin freely around the base. In the base there may be interior dividing walls which may run between the outer wall of the inner cylinder and the interior walls of the main base cylinder. These divider walls may be the same thickness as other areas and be the same height as the walls of the base and the interior cylinder. There may be 4 walls creating 4 separate chambers within the base. These chambers may hold three different items related to the administration of administering first aid to minor scrapes, wounds or cuts and one chamber may be left empty. The empty chamber may be where the dial rests when the device is stored and not in use. The other chambers may contain approximately 20 individually wrapped cleaning wipes, approximately 20 individually wrapped disinfectants wipes (such as antibacterial), and approximately 20 individually wrapped bandages. The individually wrapped items may be approximately ⅞ of an inch tall, approximately 3 inches long and approximately 1/16 inch thick. Each individually wrapped item may be vertically stacked so that the flat surface of the item may be pressed towards the outer edge of the base wall. The items may be stacked horizontally within each chamber in this fashion. The height of the items may be slightly less than the height of the base wall. For purposes of keeping pressure on the items towards the external wall a piece of foam or similar material may be inserted into the chamber so that it fits the space of the chamber but may be mendable or otherwise spongy such that the items can squeeze between this material and the outer wall and have constant pressure outward. There may be strategically placed openings in the outer wall in the three chambers such that the interior items can be extracted once the base lid is permanently attached. These strategically placed openings may be smaller in size than the items so, to extract the items, a user can grab the desired item such as a bandage but has to apply pressure to remove the item from the device. Once the items are in place, a permanent base lid may be attached to the base sealing in the items. The permanent base lid may likely be made of the same material as the base and be the same thickness, diameter, and circumference as the base. The center of the base lid may have a hole or opening that it is aligned with the opening in the base’ center cylinder. This base lid may have instructions for the user on how to utilize the device and items within the device. The instruction on the permanent base lid may align with the items located in the device below. The bottom of the base may have an additional exterior layer about 1/16 inch thick of a tacky material such as rubber that encompasses the exterior bottom of the base and curves up about 118th of an inch around the exterior bottom side of the base. The purpose of the tacky material is to allow a user to grip the base while using the device and either spinning the attached spinning lid or extracting the items. Additionally, the tacky material allows the device base to remain stationary when the user elects to use the device while it rests upon a flat surface such as a table.

The spinning lid component may be made of plastic or similar material as the base with similar thickness as the base. The spinning lid component may attach to the base once the base components are installed and the permanent base lid may be attached to the base. The spinning lid with bowl feature may rotate around the base and expose sections of the base in a systematic order such that the items of the base can be extracted. The design of the spinning lid includes a center divot or bowl feature. The bowl feature within the spinning lid may be the mechanism for attaching or combining the base and the spinning lid and acts as an independent chamber outside of the base to hold waste from the items when utilized.

The spinning lid may be approximately 1116th of an inch thick and may be slightly larger in circumference than the base. The spinning lid may have side walls with the same thickness as other areas of the storage container and these side walls may extend over the exterior sides of the base and stop just above the tacky material built into the base when the spinning lid is attached to the base. The spinning lid may have a center cup that is cupped out so that the interior of this cup is hollow and this cup extends downward away from the spinning lid's disc area. The center cup has a floor. The depth of the spinning lid's cup is slightly deeper than the height of the base's interior cylinder walls including the permanent base lid. The circumference of the spinning lids cup feature is such that it is slightly smaller in diameter than the base's interior cylinder walls. The spinning lids bowl feature may also taper in size at the same ratio as the base's interior cylinder. The exterior base of the spinning lid's cup feature may have a protruding ledge around it such that the spinning lid's cup feature slides into the base's center cylinder and the protruding edge of the spinning lid's cup feature snaps past and into the undercut edge of the base's interior cylinder, locking the two pieces together yet allowing the spinning lid to spin freely around the base. The spinning lid may not be a complete circle, there may be a portion removed from the outer wall of the spinning lid including its sides inward until the cutout reaches the bowl feature. The cutout may be larger at the exterior of the spinning lid and may taper inward as it gets closer to the bowl feature. There may be nodules placed on the upper portion of the interior walls of the base's interior cylinder which may strategically align with holes of smaller diameter than the nodule in the exterior wall of the cup feature within the spinning lid. This nodule and hole system may temporarily fix the spinning lid in a position so that the spinning lid essentially has four strategic locations along its spin that the spinning lid cut out may stop or lock in place at these nodules thus strategically revealing 4 different sections of the base. The pressure created at these nodules may be enough that the user knows they have spun the dial to a desired location and the dial has restriction in moving but with steady pressure the user can spin through the nodule on to the next stop. These stops expose the base through the cutout feature of the spinning dial in four strategic locations, thus allowing user to extract the items from the side of the base. In this example, the four locations represent a section which is inactive, locked, or otherwise not in use, and then three other locations related to administering first aid to a minor cut or scrap: clean, apply ointment, and apply a bandage. Each section may have specific instructions on the base lid for each revealed base location. The instructions may identify items that are currently accessible and a visual indicator of how to proceed. An example of a visual indicator may include an arrow for the direction to start spinning and an arrow towards the center indicating the center bowl is for trash. The exterior walls of the spinning lid may have tacky material so the user can easily grip and spin the device. The spinning lid cup feature may have a permanently attached slotted flexible lid made out of foam that allows a user to push trash generated from the items, such as bandage wrappers, into the cup past the lid while keeping the wrappers from coming out.

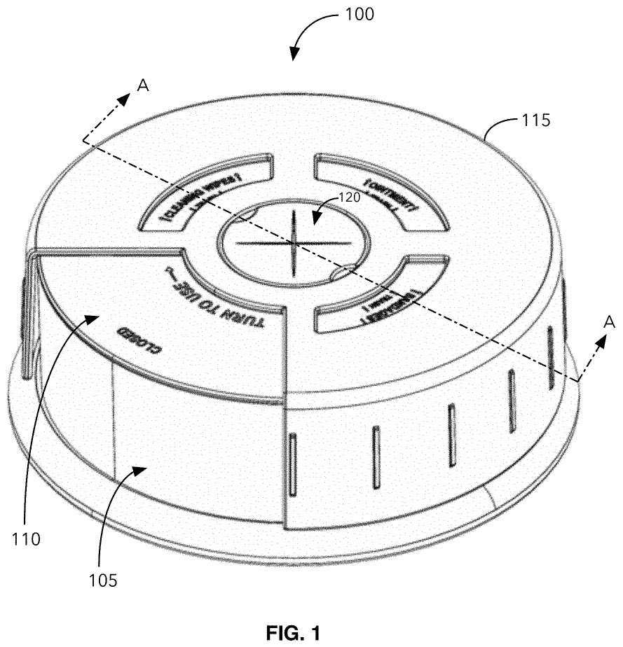

FIG. 1 illustrates an example storage container 100 with a base 105, an intermediate lid 110, a spinning lid 115, and a trash cover 120. The storage container 100 may be used to hold various items in separate chambers while making each chamber accessible to the user in a particular order. For instance, the storage container 100 may serve as a first aid kit, storage container for hobby items, jewelry-making kit, etc.

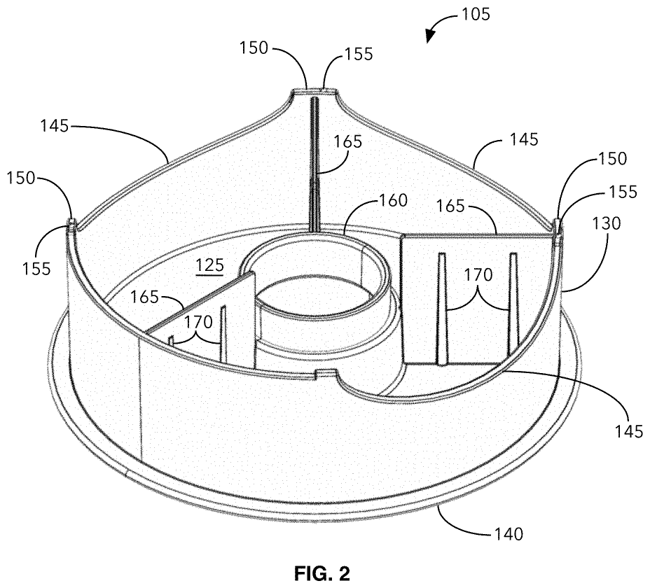

FIG. 2 illustrates an example base 105. The base 105 includes a floor 125, a base wall 130, and a center chamber 135.

The floor 125 defines the bottom surface of the base 105. The floor 125, therefore, may be the part of the base 105 that rests on a table or other surface. In some possible implementations, the floor 125 includes a grip 140 formed from a tacky material such as rubber. The grip 140 may prevent the base 105 from slipping when placed on a smooth surface such as a table, especially when the storage device is in use (e.g., the spinning lid 115 is being rotated).

The base wall 130, which extends upwardly from the floor 125, encircle the base 105 about its outer periphery. The base wall 130 may define cutouts 145 that make different sections of the base 105 more accessible to the user. The cutouts 145 need not have the same shape. For instance, at least one cutout may be deeper than one or more other cutouts 145. The base wall 130 may further include peaks 150 between the cutouts 145. A nodule 155 may be located at the top of each peak. As described in greater detail below, the nodule 155 may be used to create stopping points for the rotation of the spinning lid 115.

The center chamber 135 is defined by a cylindrical tube 160 that extends perpendicularly from the floor 125. In some possible approaches, the cylindrical tube 160 includes multiple segments of different diameters. Moreover, the segments of the cylindrical tube 160 may be tapered slightly so that the portion of the segments nearest the floor 125 are wider than the portion of the segments nearest the spinning lid 115 and intermediate lid 110. The center chamber 135 can receive portions of the spinning lid 115 and intermediate lid 110 when the storage container 100 is assembled. Chamber walls 165 extend from the center chamber 135 to the base wall 130. The chamber walls 165 separate the base 105, and therefore the storage container 100, into sections. Each section can hold different items. In some possible implementations, the sections are approximately the same size. One or more sections, however, may be of a different size than one or more other sections. For instance, the size of each section may be based on the sizes of the items contained within the section, the size of the gap (discussed below) in the spinning lid 115, or a combination of both. Each chamber wall 165 may include one or more spines 170 that provide structural support to the chamber walls 165.

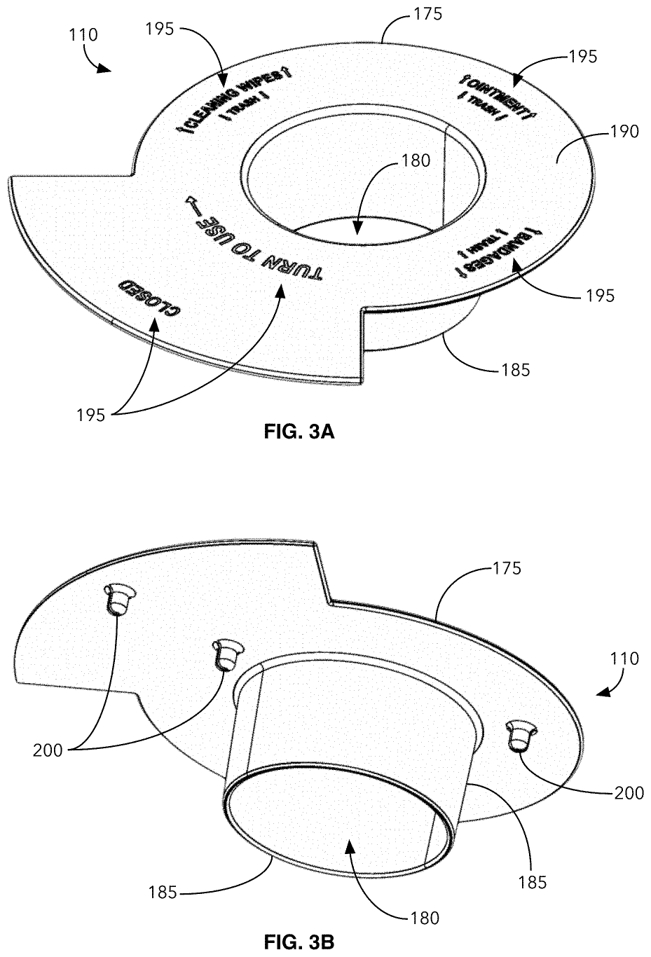

Referring now to FIG. 3A, the intermediate lid 110 defines a top piece 175 with an opening 180 in the middle. A generally cylindrical appendage 185 may be integrally formed with the top piece 175 and extend from a bottom surface of the top piece 175. A center of the appendage 185 may align with the opening 180 in the top piece 175. The appendage 185 may have a generally cylindrical shape that substantially matches an inside surface of the cylindrical tube 160 of the base 105 to, e.g., allow the appendage 185 to be inserted into the cylindrical tube 160 when the storage container 100 is assembled.

The edges of the intermediate lid 110 may be rounded to match the circular configuration of the base wall 130 shown in FIG. 2. While a unitary piece, different parts of the intermediate lid 110 may extend different radial distance when measured from the center of the opening 180. For instance, as shown in FIG. 3, most of the intermediate lid 110 has a smaller radial length than the rest of the intermediate lid 110. The different radial lengths may permit the storage container 100 to be closed by substantially covering an opening 180 in the spinning lid 115. That is, the part of the intermediate lid 110 with the longer radial dimensions may cover enough of the base 105 to prevent items from falling out during, e.g., transportation or tipping of the storage container 100. As such, the part of the intermediate lid 110 with the smaller radial length may align with the sections of the base 105 where items may be stored.

A top surface 190 of the top piece 175 may include markings 195. The markings 195 may provide instructions for using the storage container 100. The example markings 195 shown in FIG. 3 are for a storage container 100 used as a first aid kit. The markings 195 indicate how the spinning lid 115 should be rotated about the base 105, which items are contained in each section of the base 105, where trash should be placed, and the position of the spinning lid 115 when the storage container 100 is not in use (e.g., “closed”). The markings 195 may be in order in which the items will be used. For instance, returning to the example where the storage container 100 is used as a first aid kit, the markings 195 indicate that the items should be used in the following order: cleaning wipes, ointment, and bandages.

As shown in FIG. 3B, a bottom surface of the top piece 175 may define clips 200. Each clip 200 may attach to one of the chamber walls 165 extending between the center chamber 135 and the base wall 130. Attaching the clips 200 to the chamber walls 165 may prevent the intermediate lid 110 from rotating, especially when the spinning lid 115 is being rotated by the user.

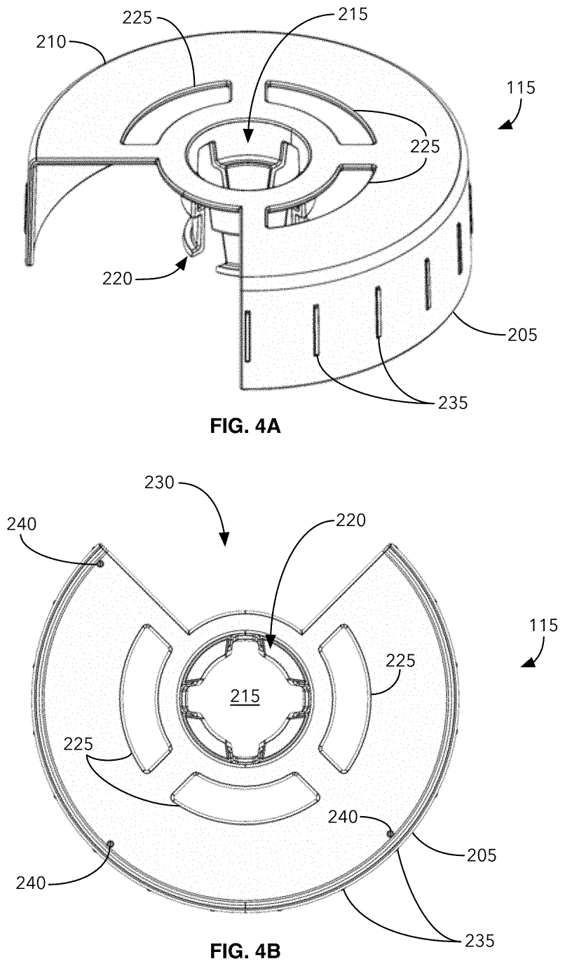

FIGS. 4A and 4B illustrate an example spinning lid 115. The spinning lid 115 may be a single piece with a side wall 205 extending from a central element 210 toward the base 105 when the storage container 100 is assembled. The central element 210 may define an aperture 215 that aligns with the opening 180 in the top piece 175 of the intermediate lid 110 and the center chamber 135 of the base 105. The spinning lid 115 may further include a coupler 220 extending from a bottom of the central element 210 and in the same direction as the side wall 205. The coupler 220 may be shaped to attach to the appendage 185 of the intermediate lid 110, the cylindrical tube 160 of the base 105, or both. The central element 210 may further define windows 225. The windows 225 allow certain markings 195 on the top surface 190 of the intermediate lid 110 to be viewable at certain times, such as when the spinning lid 115 is rotated to certain positions relative to the intermediate lid 110. The spinning lid 115 may further define a gap 230. When the storage container 100 is fully assembled and the spinning lid 115 rotated to a particular position, the gap 230 may permit a user access to one or more sections of the base 105. That is, the gap 230 may allow a user to reach into the section to retrieve whatever item may be located in that section. Vertical slats 235 spaced along the slide walls may make it easier for a user to rotate the spinning lid 115 relative to the base 105.

FIG. 4B is a bottom view of the spinning lid 115. As shown in FIG. 4B, the bottom surface of the central element 210 defines notches 240 for receiving the nodules 155 atop the peaks 150 of the base wall 130. When the user rotates the spinning lid 115 to predetermined positions relative to the base 105, the notches 240 will catch the nodules 155 to hold the spinning lid 115 in place. The predetermined positions align with the sections of the base 105. That is, when the spinning lid 115 is in one of the predetermined positions, the user will either be able to access the items in a particular section of the base 105 via the gap 230 or the gap 230 will expose the top surface 190 of the intermediate lid 110, indicating that the storage container 100 is “closed.”

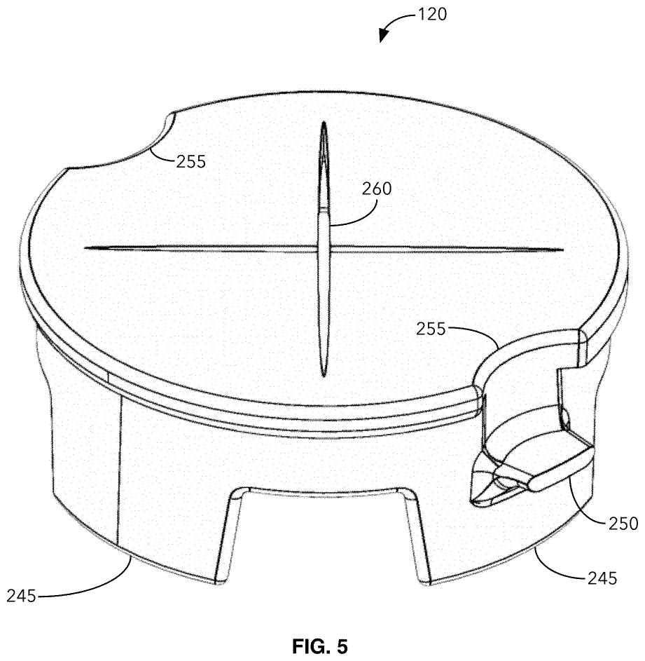

FIG. 5 illustrates an example trash cover 120. The trash cover 120 may be inserted into the aperture 215 of the spinning lid 115 when the storage container 100 is assembled. The trash cover 120 may include vertical limbs 245 that extend into the aperture 215 of the spinning lid 115. One or more of the limbs 245 may include an integrally formed tab 250. The tab 250 may hold the trash cover 120 in place inside the spinning lid 115. The tab 250 may be located adjacent to a recess 255 in the trash cover 120 to make it easier for the user to pinch the trash cover 120 to release the tab 250 from inside the spinning lid 115 if, e.g., the user wishes to remove the trash cover 120. In one possible implementation, the top of the trash cover 120 is a flexible material, such as rubber or a flexible plastic, that defines slits 260. The slits 260 allow the user to insert trash into the center of the storage container 100 while minimizing the risk that trash will fall out when, e.g., the storage container 100 is being transported.

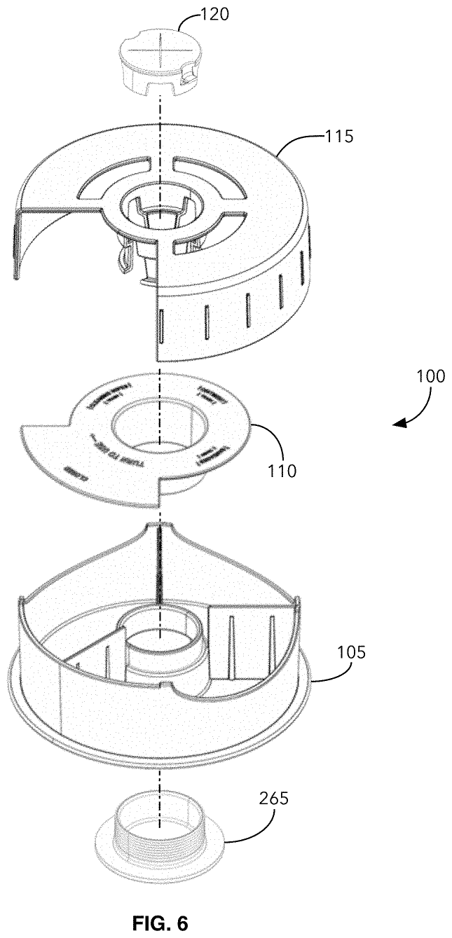

FIG. 6 illustrates an exploded view of the storage container 100. Specifically, FIG. 6 shows how the base 105, intermediate lid 110, spinning lid 115, and trash cover 120 connect to form the storage container 100. Also shown in FIG. 6 are the grip 140 and a plug 265. The grip 140 defines a hole for receiving the plug 265, which may be inserted through a corresponding hole in the floor 125 of the base 105. The plug 265 may be threaded to attach to corresponding threads on the inside surface of the cylindrical tube 160. With the plug 265 inserted and screwed into the base 105, trash placed into the center of the storage container 100 will remain in place. That is, it will not fall through holes in the floor 125 and/or grip 140 of the base 105. When the user wishes to empty the trash receptacle, the user may unscrew or otherwise remove the plug 265 from the cylindrical tube 160 to expose the hole, empty the trash from inside the center of the storage container 100, and screw in or otherwise return to the plug 265 to the cylindrical tube 160 to fill the holes. In some possible approaches, an anchor may be used to keep the plug 265 attached to the base 105 while still allowing the plug 265 to be inserted into and removed from the hole in the base 105.

In some possible approaches, the plug 265 may be omitted. In that case, the floor 125 of the base 105 may extend completely under the cylindrical tube 160. In that instance, trash may be collected within the cylindrical tube 160. In another possible implementation, the appendage 185 of the intermediate lid 110 or the coupler 220 of the spinning lid 115 may be closed at the bottom to catch refuse. In each of those instances, the trash may be emptied by, e.g., removing the trash cover 120 and pouring trash out from the center of the storage device.

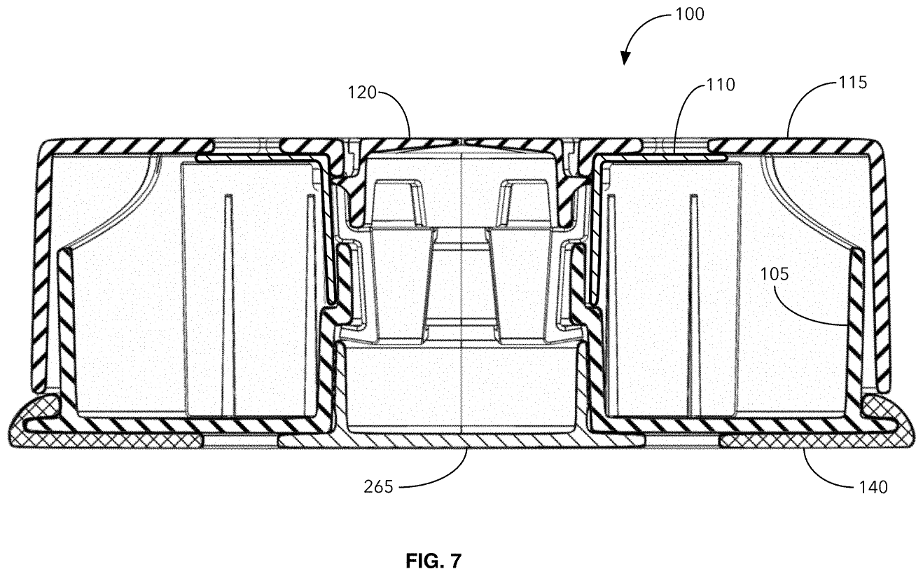

FIG. 7 is a cross-sectional view of the assembled storage container 100 taken along the line A-A of FIG. 1. As shown, the center chamber 135 of the base 105, the opening 180 of the intermediate lid 110, and the aperture 215 of the spinning lid 115 are coaxially aligned to define a trash receptacle at the center of the storage container 100. The elements of the storage container 100 fit together in a way that permits rotation of the spinning lid 115 relative to the base 105 while keeping the trash receptacle at the center of the storage container 100 accessible at all times. FIG. 7 further shows how the tab 250 of the trash cover 120 is held into place relative to the spinning lid 115 without inhibiting rotational motion of the spinning lid 115. Moreover, FIG. 7 illustrates how the coupler 220 of the spinning lid 115 snaps into the cylindrical tube 160 of the base 105. That is, as previously discussed, the cylindrical tube 160 may have different diameters to create a step. The coupler 220 of the spinning lid 115 may clip into the cylindrical tube 160 at the step. When connected, the spinning lid 115 may be able to rotate relative to the base 105 but will not be removed unless the user unclips the coupler 220 from the cylindrical tube 160. FIG. 7 further illustrates that the intermediate lid 110 is sandwiched between the base 105 and the spinning lid 115.

Also shown in FIG. 7, the nodules 155 of the base 105 align with the notches 240 in the spinning lid 115. The alignment of the nodules 155 with the notches 240 will inhibit the spinning lid 115 from rotating until the user provides a sufficient rotational force to release the nodules 155 from the notches 240. As such, the spinning lid 115 will stay in place until the user wishes to access items in a different segment of the base 105.

It is to be understood that the above description is intended to be illustrative and not restrictive. Many embodiments and applications other than the examples provided would be apparent upon reading the above description. The scope should be determined, not with reference to the above description, but should instead be determined with reference to the appended claims, along with the full scope of equivalents to which such claims are entitled. It is anticipated and intended that future developments will occur in the technologies discussed herein, and that the disclosed systems and methods may be incorporated into such future embodiments. In sum, it should be understood that the application is capable of modification and variation.

All terms used in the claims are intended to be given their ordinary meanings as understood by those knowledgeable in the technologies described herein unless an explicit indication to the contrary is made herein. In particular, use of the singular articles such as “a,” “the,” “said,” etc. should be read to recite one or more of the indicated elements unless a claim recites an explicit limitation to the contrary.

The Abstract is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

1. A storage container comprising:
a base having a plurality of sections and defining a center chamber;
a spinning lid disposed on the base, wherein the spinning lid defines an aperture; and
an intermediate lid disposed between the base and the spinning lid and defining an opening, wherein the center chamber, aperture, and opening are coaxially aligned to define a receptacle.

2. The storage container of claim 1, wherein the base includes:
a floor,
base wall extending from the floor; and
a cylindrical tube defining the center chamber.

3. The storage container of claim 2, wherein the base wall includes a plurality of peaks spaced from one another, wherein each peak includes a nodule.

4. The storage container of claim 3, wherein the base wall defines cutouts between each of the plurality of peaks.

5. The storage container of claim 2, wherein the cylindrical tube extends perpendicularly from the floor and includes multiple segments, each having a diameter.

6. The storage container of claim 2, wherein the base includes a plurality of chamber walls extending from the cylindrical tube to the base wall.

7. The storage container of claim 1, wherein the spinning lid includes a coupler extending from a bottom surface of a central element.

8. The storage container of claim 7, wherein the coupler includes clips for attaching to the base.

9. The storage container of claim 7, wherein the central element defines a plurality of windows.

10. The storage container of claim 7, wherein the spinning lid defines a gap to make at least one of the plurality of sections of the base available to a user.

11. The storage container of claim 7, wherein the spinning lid includes a side wall extending from the central element, wherein the side wall includes a plurality of slats.

12. The storage container of claim 1, wherein the intermediate lid includes a top piece defining the opening and a generally cylindrical appendage extending from a bottom of the top piece.

13. The storage container of claim 12, wherein the top surface includes markings.

14. The storage container of claim 1, further comprising a cover disposed on at least one of the base, the spinning lid, and the intermediate lid over the receptacle.

15. The storage container of claim 14, wherein the cover includes a plurality of slits.

16. The storage container of claim 1, further comprising a plug disposed on the base and extending into the center chamber.

17. A storage container comprising:
a base having a floor, a base wall extending from the floor, and a cylindrical tube defining a center chamber, and at least one chamber wall separating the base into a plurality of sections;
a spinning lid disposed on the base, wherein the spinning lid includes a coupler defining an aperture, wherein the coupler clips to the cylindrical tube of the base; and
an intermediate lid disposed between the base and the spinning lid and defining an opening, wherein the center chamber, aperture, and opening are coaxially aligned to define a receptacle,
wherein the base wall includes a plurality of peaks spaced from one another, and wherein each peak includes a nodule, and
wherein a bottom surface of the spinning lid defines a notch for engaging the nodule on at least one of the plurality of peaks.

18. The storage container of claim 17, further comprising a cover disposed on at least one of the base, the spinning lid, and the intermediate lid over the receptacle.

19. The storage container of claim 17, further comprising a plug disposed on the floor of the base and extending into the center chamber.
